(19) United States
(12) Patent Application Publication
(10) Pub. No.: US 2022/0362529 A1
(43) Pub. Date: Nov. 17, 2022

(54) MULTI-MODALITY SKIN TREATMENT DEVICE

(71) Applicant: CARISMED Corporation, Irvine, CA (US)

(72) Inventor: Serge Castro, Irvine, CA (US)

(21) Appl. No.: 17/743,110

(22) Filed: May 12, 2022

Related U.S. Application Data
(60) Provisional application No. 63/187,656, filed on May 12, 2021.

(51) Int. Cl. A61M 37/00; A61N 5/06; A61N 1/40; A61N 1/32; A61M 35/00; A61M 1/00

(52) U.S. Cl. CPC A61M 37/0015; A61N 5/0616; A61N 1/40; A61N 1/327; A61N 1/325; A61M 35/003; A61M 1/85; A61M 2037/0007; A61M 2037/0023; A61M 2205/051; A61M 2205/055; A61M 2205/054; A61N 2005/0651

(57) ABSTRACT

A skin treatment device includes a control unit and a hand tool. The hand tool includes a body, a wiper head, and a needle roller head. The wiper head includes a rotatable wiper structure including one or more wiper blades. The needle roller head includes needle roller structure. The skin treatment device provides a multi-step process including desincrustation, skin transdermal delivery of topical agents, serums, and the like, microdermabrasion, and fractional micro-channeling application.

### CROSS REFERENCE TO PREVIOUSLY FILED APPLICATIONS

The present application claims the benefit of priority to U.S. Application No. 63/187,656 filed May 12, 2021, the entire contents of which are incorporated herein by reference.

### BACKGROUND OF THE INVENTION

The present subject matter relates generally to skin treatment devices that provide desincrustation followed by skin transdermal delivery treatment for a large and diverse scope of treatments/applications. Specifically, the present subject matter relates to a single skin treatment device that provides a multi-step process including desincrustation, skin transdermal delivery of topical agents, serums, and the like, microdermabrasion, and fractional micro-channeling application.

Skin debris and makeup remover devices are typically solutions or hand-held devices designed to be used by a person on their own face rather than for professional use. These inefficient methods usually use prefabricated fabric pads impregnated with cleansing liquid, tissues, or wet wipes to remove makeup powder and/or other buildup from the skin. In one example, pads are held in place on manually rotating tips, which the user applies across the face to scrub material therefrom.

Skin professionals and aestheticians typically use tissues and wipes with cleansing agents to manually and more efficiently remove debris such as skin pollutants, makeup and other impurities from a client's or patient's face. The procedure is completely manual and therefore is time-consuming, and is generally limited to the removal of the makeup/debris and is only partially effective. In addition, the cleaning procedure is typically followed by a second manual procedure for the application of a skin hydrating cream onto the face and neck to prevent dryness from the cleansing agents of the cleaning procedure.

In addition, various skin treatment methods may also be used that apply serums or pharmaceutical agents to the patient's skin. In order for the serum to penetrate the skin and be most effective, skin care professionals typically recommend microdermabrasion to remove the pollutants and debris from the skin prior to the application of the serums and/or the use of skin rollers after the application of the serum on the skin. However, many of these methods require separate skin transdermal delivery treatment devices and numerous sessions for achieving both the effective cleansing of the skin debris and the application and penetration of the serum into the skin.

Based on research conducted in connection with the development of this invention the main reasons for the difficulty for serums to penetrate the skin, is the fact that

One of the main functions of skin is to maintain a structural and functional barrier to external environmental challenges to the patient, making the skin difficult to penetrate for serum application. This barrier needs to be breached to allow the passage of drugs, biologic molecules, and cosmeceuticals for skin improvements. The combined application of microdermabrasion (MDB) and minimal fractional penetration by micro-needling (MN) along with their array of active topical agents has the potential to transdermally deliver safe and effective treatments for multiple skin conditions that include rhytids, acne, dehydrated skin, hyperpigmentation, striae, actinic keratoses, and scars.

Innovative equipment combining microdermabrasion and microneedling on the market, such as the MIRApeel MD device, temporarily compromise the lipid barrier in the outer layer of the stratum corneum of the skin as well as create reversible micro-clefts for the passive passage of macromolecules from cosmeceuticals, drugs, platelet-rich particles, and bioactive secretomes. Applying the concepts and the techniques of such systems to facial skin treatments appears to achieve a safe procedure in producing minimal injury to transdermally deliver through the lipid barrier and rapid recovery by 48 hours after combined MDB and MN treatments.

Accordingly, to meet these varied objectives, a need exists for an automated multi-modality skin treatment device that provides desincrustation and pollutant removal while also providing a transdermal delivery of serums and other agents as described herein.

### BRIEF SUMMARY OF THE INVENTION

To meet the needs described above and others, the present disclosure provides a skin treatment device that provides a desincrustation treatment of the pores of the skin and consecutively provides numerous applications to transdermally deliver active topical agents into the top layers of the skin. Desincrustation is defined here as the chemical, galvanic and/or mechanical process to achieve both the deep cleansing of the skin, remove debris, facial make-up and environmental pollutants, and to soften and emulsify sebum and keratin in the follicle.

Specifically, the present subject matter relates to a device that combines multistep applications, starting with the desincrustation process that is followed by skin transdermal delivery of serums and other topical agents using a combined and controlled microdermabrasion and fractional microchanneling application. The transdermal delivery is used to infuse active topical agents, and is tentatively augmented by innovative integrated methods of microneedles vibration and electrophoresis to achieve better and deeper penetration.

The skin treatment device includes a table top control unit and a hand tool that may accommodate a plurality of heads for treatment. The hand tool may be equipped with a rotating wiper head, a roller head including needles intended to be rolled across the user's skin to create microchannels into the top layers of the skin to work a medication or other serum into the user's skin, or comprised of both the rotating wiper head and the roller head including needles, one of each head for interchangeable use. The hand tool may also incorporate galvanic and iontophoresis modules and use alternative motors for added micro-vibration of the needles to enhance blood flow and improve serum penetration.

In one example, an automated multi-modality desincrustation and skin transdermal delivery treatment device includes a hand tool having a base, a body, and a head. The base is connected to the control unit through a connector. The body includes a casing enclosing the mechanisms described further below. The head is located at the distal end of the body and includes mechanisms adapted to perform the advantageous functions described herein. Two main embodiments are described herein, but it is understood that the head may be adapted for use with interchangeable heads of varying utility.

In one example, a head includes a rotating wiper, a flexible tip of adjustable grits, roughness level and texture for contacting the patient's face, a plurality of nozzles and a plurality of drains. The nozzles are in fluid communication with one or more supply lines and the drains are in fluid communication with a vacuum line and/or a vacuum chamber. The rotating wiper may be, for example, a replaceable silicone rubber wiper whose shape, roughness level and texture may correspond to the skin type and the one or more treatments to be implemented.

In this example, the body includes a regulated supply line including a feed pipe, a feed control valve and a feed control button. The body further includes a motor and gearbox powering a wiper drive, which connects to the rotating wiper. A vacuum pipe is associated with the head's vacuum line and/or a vacuum chamber. In this example the body includes a power switch for turning on and off the hand tool and a head contact switch, which activates the gearbox when the rotating wiper is in contact with a treatment surface.

The hand tool may include, for example, a detachable base with electric and hydraulic connectors for mating with the elements in the body. For example, the base may include a base feed pipe and a base vacuum pipe, and the body may include a body feed pipe and a body vacuum pipe, with each of the base and body feed pipes and the base and body vacuum pipes interconnecting along the interfacing surfaces thereof. In one contemplated example, the detachable base includes a connector for connecting the hand tool to a control base.

The control base may include hydraulic controls and, based on the elements of the embodiment described above, the hydraulic controls may include: a pressure pump for the supply line, a pressure sensor and pressure relief control associated with the supply line; and a vacuum pump for the vacuum pipe, a vacuum filter and a plurality of vacuum level controls. In addition, the base may include various supply chambers for supplying various materials for the hand tool, such as, for example, disinfectant, facial cleaner, treatment serum, distilled water, etc. The control base may further include, for example, a steam generator and temperature sensor associated with the distilled water supply.

It is understood that the hydraulic controls (and/or associated electronic controls) may be entirely contained within the body to provide a portable handheld device. In other example, such hydraulic controls may reside in the control base providing a larger less portable solution. In such cases, the body may be considered an adapter for mating the chosen functional head to the hydraulics and electronics resident in the control base.

In a second example, the head may include a plurality of outwardly facing needles that may be made of different materials and may be adjustable in needle size, build, and/or shape, arranged along an outer surface of a rotating ball or cylinder. The head may be used to percutaneously deliver medication or other serums to the user's epidermis and/or dermis. Such delivery may be made by application of the serum prior to the use of the needle roller or simultaneously with the use of the needle roller. For example, one skilled in the art will understand that the hydraulic controls described immediately above may be used with a needle roller head to deliver medication or other serums to the user's skin surface to be rolled into the skin by the needle roller head (by delivering the serum to the surface of the needle roller head or to the user's skin). In addition, the hydraulic controls may be integrated with needles, such that serum delivery occurs through one or more of the needles themselves.

In a third example, the needle roller head may include an integrated method to achieve microvibration (MVT) of the needles to increase blood flow, help to release muscle stiffness, and improve serum absorption.

In a fourth example, the needle roller head may include an iontophoresis module to help molecules get transported across the stratum corneum by electrophoresis and electro-osmosis. The electric field also increases the permeability of the skin, to achieve an even better and deeper penetration.

The control base may further include electronic controls, including, for example, a controller, a power supply, a display screen and control module, as well as various A/D inputs, digital inputs and drivers for the various pumps and drivers.

In other examples, the automated multi-modality desincrustation and skin transdermal delivery treatment device may include, for example: a heat generating mechanism using a radio frequency oscillator module; a phototherapy LED module; as well as other modules for galvanic desincrustation, microvibration, and/or chemical pollutant removal modules, and advanced skin transdermal delivery treatment modalities using wet-microdermabrasion, micro-channeling and electrical iontophoresis.

The automated multi-modality desincrustation and skin transdermal delivery treatment device is intended for use by a professional at a spa, an aesthetic salon, a doctor's office, by a hair-dresser, on movie and TV sets, and/or any location where desincrustation is being performed by a professional skin specialist. The skin treatment device may provide safe and effective treatments for multiple skin conditions such as rhytids, acne, dehydrated skin, hyperpigmentation, striae, actinic keratoses, and scars, and/or purely aesthetic skin rejuvenation, texture improvement, oxygenation and/or skin toning procedures are offered. The automated multi-modality desincrustation and skin transdermal delivery treatment device is especially useful in situations in which pollutant materials and residues must be removed from the client/patient face (and neck) just prior to a skin procedure or skin treatment.

When using the wiper head adapted for pollutant removal, the automated multi-modality desincrustation and skin transdermal delivery treatment device is unique in being quick and providing a thorough and efficient facial scrub. When activated and in contact with the skin, the automated multi-modality desincrustation and pollutant removal and skin transdermal delivery treatment device applies one or more cleaning serums to the area to be cleansed. The rotating wiper then brushes the cleansing serum together with the makeup buildup, greasy materials residues, and other debris off the skin, sucking them away from the skin through the device and clearing-out the facial pores from pollutants and skin debris. When operating, the vacuum also slightly sucks-in the skin surface which is in contact with the flexible tip of the device to improve contact and efficacy of the procedures and treatments.

The automated multi-modality desincrustation and skin transdermal delivery treatment device may be controlled using pre-programmed parameters for safest use. The device is designed for multiple uses every hour, on numerous clients/patients, and may be adapted, for example, to automatically end each procedure with a comprehensive self-disinfecting cycle to avoid the risk of cross-contamination, as administered in a professional environment.

As described above, when using the needle roller head, the skin transdermal delivery treatment device may include at least one nozzle for dispensing a liquid or a gel, such as a serum. The nozzle may dispense the serum directly onto the roller with needles, the serum penetrating the skin when perforation occurs. Alternatively, the nozzle may be in fluid communication with the roller such that the serum may be dispensed directly through one or more of the needles to penetrate the skin and dispense the serum directly into the top layer of the skin. The nozzle may be in fluid communication with one or more supply lines connected to a serum source. In addition, the head may be in direct fluid communication with a vacuum line and/or a vacuum chamber or through a controlled serum dispenser or cartridge attached to the treating handpiece or the delivery system in other ways.

In one embodiment of the present invention, a skin treatment device includes a control unit and a hand tool. The control unit includes a control screen and a plurality of reservoirs. The hand tool connected to the control unit includes a body with a base end and a head end, the body includes a feed control valve controlling flow through a body feed line, a body vacuum line, and a motor. The body further includes a control button configured to actuate the feed control valve. The skin treatment device also includes a wiper head and a needle roller head, each configured to selectively attach to the head end of the body.

The wiper head includes a wiper head casing including a fluid duct and a vacuum duct, wherein, when the wiper head is attached to the head end of the body, the liquid duct is in fluid communication with the body feed line and the vacuum duct is in fluid communication with the body vacuum line and vacuum duct, a rotatable wiper structure including one or more wiper blades, wherein, when the wiper head is attached to the head end of the body, the rotatable wiper structure is operably driven by the motor, and one or more nozzles in fluid communication with the plurality of reservoirs in the control unit through the body feed line and fluid duct.

The needle roller head includes a roller head casing including a fluid duct and a vacuum duct, wherein, when the wiper head is attached to the head end of the body, the liquid duct is in fluid communication with the body feed line and fluid duct and the vacuum duct is in fluid communication with the body vacuum line and vacuum duct, a needle roller structure, and one or more nozzles in fluid communication with the plurality of reservoirs in the control unit through the body feed line.

In some embodiments, the wiper blades are silicone. In some embodiments, the wiper blades include an abrasive contact surface. The wiper head may include one or more LEDs electrically coupled to a power source in the control unit through a power line in the hand tool. The wiper head may include a radio-frequency generator electrically coupled to a power source in the control unit through a power line in the hand tool. The wiper head may also or alternatively include an electric field generator electrically coupled to a power source in the control unit through a power line in the hand tool, the electric field generator including a main electrode and one or more reference electrodes.

In some embodiments, the needle roller head includes one or more LEDs electrically coupled to a power source in the control unit through a power line in the hand tool. The needle roller head includes a radio-frequency generator electrically coupled to a power source in the control unit through a power line in the hand tool. The needle roller head may also or alternatively include an electric field generator electrically coupled to a power source in the control unit through a power line in the hand tool, the electric field generator including a main electrode and one or more reference electrodes.

In some embodiments, one or more of the wiper head and the needle roller head includes one or more of: one or more LEDs electrically coupled to a power source in the control unit through a power line in the hand tool; a radio-frequency generator electrically coupled to a power source in the control unit through a power line in the hand tool; and an electric field generator electrically coupled to a power source in the control unit through a power line in the hand tool, the electric field generator including a main electrode and one or more reference electrodes.

In still further embodiments, the control unit includes a controller and memory, the memory storing program instructions that, when processed by the controller cause the controller to: request a user selection of a treatment modality through the control screen; present instructions for use of the hand tool through the control screen; and automatically control one or more functions of the hand tool according to the selected treatment modality.

For example, the instructions may present a guide for a skin cleansing modality including instructing the attachment of the wiper head to the body and the control unit adjusts a vacuum power applied through the wiper head and provides a flow of cleansing material through the one or more nozzles while rotating the wiper blades. The control unit may then subsequently provide water or steam through the one or more nozzles while rotating the wiper blades. The control unit may then subsequently provide a galvanic gel through the one or more nozzles while rotating the wiper blades and generating an electric field through the electric field generator.

In another example, the instructions present a guide for a transdermal delivery treatment modality including instructing the attachment of a wiper head including an abrasive contact surface to the base and the control unit adjusts vacuum power and provides a flow of salicylic acid serum through the one or more nozzles while rotating the wiper blades. The control unit subsequently may then instruct the attachment of the needle roller head, adjusts vacuum power, and delivers a selected therapeutic serum through the one or more nozzles. The control unit may then subsequently instruct the application of a bio cellulose mask and activates the one or more LEDs.

In a still further example, the instructions present a guide for a transdermal delivery treatment modality including instructing the attachment of a wiper head including an abrasive contact surface to the base and the control unit adjusts vacuum power and provides a flow of salicylic acid serum through the one or more nozzles while rotating the wiper blades. The control unit subsequently delivers a hydrating serum through the one or more nozzles while rotating the wiper blades. The control unit may then subsequently activate the electric field generator or the radio-frequency generator.

An advantage of the automated multi-modality desincrustation and skin transdermal delivery treatment device provided herein is that it incorporates all phases of a skin treatment, sequentially and based on proven skin protocols, beginning from the cleaning of the face and neck (and décolleté too), flushing out all the makeup greasy large grain powder, debris materials and residues, then moving to steaming the areas to deeply cleanse the pores and helping with the toning. Further the device may be used to perform then a more aggressive cleaning and superficial abrasion of the top layers of the skin, at controlled roughness levels and diverse abrasive depths depending on the patients' skin texture and other conditions, to remove some of the dead cells, and automatically apply beneficial cosmeceutical serums or gels to the skin.

Another advantage of the automated multi-modality desincrustation and skin transdermal delivery treatment device provided herein is that it tremendously enhances improvements to skin conditions and augments the efficiency of the initial pollutant removal application by consecutively using an efficient transdermal delivery method to add fractional penetration by micro-needling of an array of active topical agents that has the potential to deliver safe and effective treatments for multiple skin conditions. It is the combination of surface abrasion (which is part of a superficial compromising of the skin to facilitate delivery of active ingredients that promote skin treatment and healthy skin function), and microneedle channeling which promotes greater delivery of actives during application and a superior reduction in transepidermal water loss, as the skin responds to the treatment. Further the micro-channeling device may be used to perform a more aggressive treatment, with microneedles penetrating deeper into the skin, at depths ranging from 0.20 mm and up to 2 mm to stimulate collagen production and further infiltrate beneficial cosmeceutical serums or gels to the skin.

Another advantage of the automated multi-modality desincrustation and skin transdermal delivery treatment device provided herein is it may be used to incorporate phototherapy light treatment, an exposure to blue and red light through light-emitting diodes (LED) treatments to enhance wound recovery and skin rejuvenation and/or focused RF treatment for incremental multi-sessions skin tightening, skin abrasion treatments, and for deeper warming of the skin to rejuvenate it and promote the collagen production.

A further advantage of the automated multi-modality desincrustation and skin transdermal delivery treatment device provided herein is to provide phased skin treatments in a one-box design that is compact, self-contained, electronically controlled, with an LCD screen and versatile hand tools.

Yet another advantage of the automated multi-modality desincrustation and skin transdermal delivery treatment device provided herein is in that it may be used to disinfect itself after each procedure using fluid disinfectant and hot vapors and steam to kill bacteria and viruses. The disinfecting procedure may be automatic and pre-programmed into every menu procedure.

Another advantage of the automated multi-modality desincrustation and skin transdermal delivery treatment device provided herein is that it enables an aesthetician or other skin specialist to provide a multitude of services and treatments with a single device, wherein the functions are automated and electronically controlled such that the provider may focus more on the treatment and patient and less on the procedures themselves.

Yet another advantage of the skin transdermal delivery treatment device disclosed herein is that a single device includes the ability to dispense a serum or a gel onto or into a patient's skin and also penetrate the serum into a patient's skin using the needle roller head.

Another advantage of the skin transdermal delivery treatment device is that, in some embodiments, the serum is only dispensed when the needles of the roller come into contact with the skin area to be treated. In other words, the device provides a most efficient manner of supplying serum to a treatment area without waste.

Moreover, the multi modalities of the automated multi-modality desincrustation and skin transdermal delivery treatment device are synergistic, complementing each other, when applied at once or in a short sequence, in one session, one after the other. Activating one modality together (or immediately in sequence after) another augments the effects of each individually to enhance and improve the final outcome.

As an example, a face treatment may start with a deep cleaning, desincrustation procedure, including brushing off the makeup greasy materials and soiling debris. The makeup and other pollutant buildup that has been potentially clogging the pores may, with time, even cause sebum obstruction (and may well be one of the initial causes of acne flares). The desincrustation and pollutant removal procedure may immediately be followed with a steam treatment. The steam treatment may open the pores temporarily and help purge the impurities from the skin through sweating. The steam treatment may also enhance circulation, bringing oxygen-and-nutrient-rich blood to the surface of the skin. Although each of these treatments has its own well-known, well-documented benefits, applying both in sequence may incrementally benefit the skin.

Additionally, to further beautify the skin, re-hydrate and replenish the skin with the necessary water content, to reaffirm the tone, and further enhance the synergistic benefits of the treatment programs, a hydrating treatment may be applied concurrently with LED phototherapy. At this phase, when the skin is warm and well oxygenated, the pores are open and clear from any obstructing materials or oily residues, applying a hyaluronic acid serum on top of the skin, or directly within the DE junction of the skin (dermal-epidermal junction), using the needles to create micro-channels to the dermal-epidermal junction and simultaneously infuse high grade serums directly into this tissue layer, with superficial to mild ablation of the top layers of the skin, together with a phototherapy LED blue (ultra-violet) and/or red light, may significantly boost the end-results, hydrate the skin to normal levels, and cut the typical treatment time in half, compared to existing treatment options.

Additional objects, advantages and novel features of the examples will be set forth in part in the description which follows, and in part will become apparent to those skilled in the art upon examination of the following description and the accompanying drawings or may be learned by production or operation of the examples. The objects and advantages of the concepts may be realized and attained by means of the methodologies, instrumentalities and combinations particularly pointed out in the appended claims.

### BRIEF DESCRIPTION OF THE DRAWINGS

The drawing figures depict one or more implementations in accord with the present concepts, by way of example only, not by way of limitations. In the figures, like reference numerals refer to the same or similar elements.

FIG. 1 is a perspective view of an automated multi-modality desincrustation and skin transdermal delivery treatment device.

FIG. 2 is a cross-sectional view of the hand tool of the automated multi-modality desincrustation and skin transdermal delivery treatment device shown in FIG. 1.

FIG. 3 is a cross-sectional view of a wiper head of the hand tool of the automated multi-modality desincrustation and skin transdermal delivery treatment device shown in FIG. 4 taken generally along line 3-3.

FIG. 4 is a top view of the wiper head of FIG. 3.

FIG. 5 is a cross-sectional view of the wiper head of FIG. 4 taken generally along line 5-5.

FIG. 6 is a bottom view of a rotating wiper of the wiper head of FIG. 3.

FIG. 7 is a cross-sectional view of the rotating wiper shown in FIG. 6 along line 7-7.

FIG. 8 is a top view of the rotating wiper shown in FIG. 6.

FIG. 9 is a cross-sectional view of the rotating wiper shown in FIG. 8 along line 9-9.

FIG. 10 is a top view of an alternative embodiment of a wiper head including a radio frequency oscillator.

FIG. 11 is a cross-sectional view of the wiper head shown in FIG. 10 along line 11-11.

FIG. 12 is a top view of a still alternative embodiment of a wiper head including an LED array.

FIG. 13 is a cross-sectional view of the head portion shown in FIG. 12 along line 13-13.

FIG. 14 is a top view of a roller head of the automated multi-modality desincrustation and skin transdermal delivery treatment device of FIG. 1.

FIG. 15 is a cross-sectional view of the head portion shown in FIG. 14 along line 15-15.

FIG. 16 is a schematic diagram of the hydraulics system of the automated multi-modality desincrustation and skin transdermal delivery treatment device shown in FIG. 1.

FIG. 17 is a schematic diagram of the electronics system of the automated multi-modality desincrustation and skin transdermal delivery treatment device shown in FIG. 1.

### DETAILED DESCRIPTION OF THE INVENTION

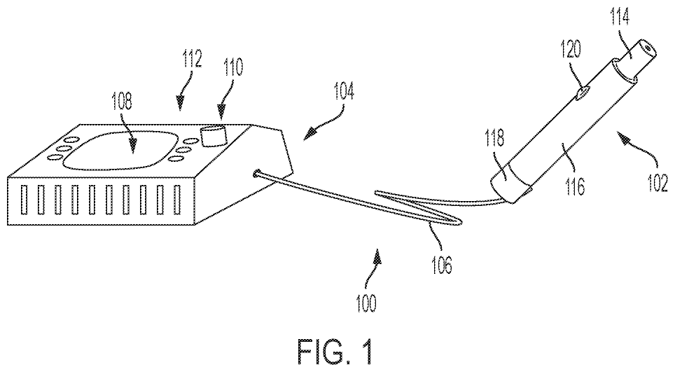

FIG. 1 illustrates an example of an automated multi-modality desincrustation and skin transdermal delivery treatment device, or skin treatment device, 100. As shown in FIG. 1, the automated multi-modality desincrustation and skin transdermal delivery treatment device 100 includes a hand tool 102 and a control base or unit 104 connected via a connector 106.

As will be described further herein, the control unit 104 may include electronic and hydraulic control systems for operating the hand tool 102, as well as a control screen 108, a hand tool cradle 110, and a supply drawer 112 for housing the materials used in the operation of the automated multi-modality desincrustation and skin transdermal delivery treatment device 100. A plurality of reservoirs is also housed within the control unit 104. The connector 106 operatively connects the control systems and materials in the control base 104 to the hand tool 102.

It is contemplated that the hand tool 102 and the control unit 104 may take numerous forms and be any of numerous configurations. For example, the control unit 104 may be configured as a personal computer operating a hydraulics rack. In another example, rather than the cylindrical form shown, the hand tool 102 may take another ergonomic hand held form.

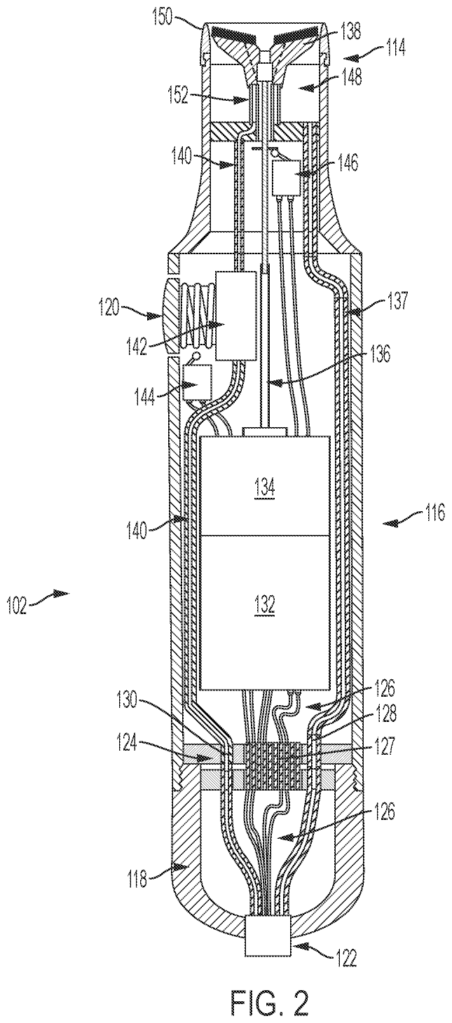

In the example shown in FIG. 1, the hand tool 102 includes a head 114, a body 116, and a detachable base 118. As further shown in FIG. 1, the hand tool 102 may include a feed control 120. A cross-sectional view of the hand tool 102 is shown in FIG. 2. The head 114 may be interchangeable between various functional heads, as described further herein. For example, in a first embodiment, the head 114 is a desincrustation or wiper head 114. In a second embodiment, the head 114 is a needle roller head 114′ for the percutaneous delivery or application of a serum. Additional or alternate heads 114 may be used with the present device 100, as will be appreciated by those skilled in the art based on the descriptions provided herein. Many of the functions of the device 100 described herein are described with reference to the wiper head 114 shown in FIGS. 2-13, though it is understood that the integration of other heads 114 will be easily understood based on the descriptions provided herein.

As shown in FIG. 2, the detachable base 118 includes a connector port 122 at one end and electric and hydraulic connectors 124 at the other end. The connector port 122 is where the connector 106 enters the hand tool 102. Then, once inside the detachable base 118, the electric and hydraulic lines 126 carried within the connector 106 separate and connect to the electric and hydraulic connectors 124. The detachable base 118 is provided to enable the hand tool 102 to be separated from the control unit 104 for cleaning or replacement without exposing the electric and hydraulic lines 126 carried within the connector 106 along the exterior of the hand tool 102. However, it is understood that in various embodiments of the automated multi-modality desincrustation and skin transdermal delivery treatment device 100, the hand tool 102 may not include a separate body 116 and detachable base 118 and that the connector 106 may be adapted to connect to the hand tool 102 in any number of ways. Additionally, in some embodiments, the device 100 may be entirely embodied within the combination of the hand tool 102 and the head 114.

As further shown in FIG. 2, the body 116 houses the corresponding electric and hydraulic connectors 124 for connection with the detachable base 118. As shown in the example shown in FIG. 2, the electric and hydraulic connectors 124 include electrical connections 127, a vacuum connection 128, and a regulated supply connection 130. Various other electric and hydraulic connectors 124 may be provided to adapt to the various controls and functions that may be provided by the hand tool 102, including those shown in the illustrated examples and those that are not.

The body 116 also houses a motor 132 and a gearbox 134 for driving a wiper drive 136. The wiper drive 136 operates a rotating wiper 138 located within the head 114 as will be described further herein. The motor 132 is a small electric motor whose configuration will be apparent to one of ordinary skill in the art. For example, the motor 132 may have limited torque to prevent the rotating wiper 138 from continuing to rotate after meeting excessive resistance. The rotating wiper 138 may contain blades that rotate and vibrate at a high frequency. Alternatively, or in addition to, the rotating wiper 138 may produce an eccentric motion of the head 114 that results in a pleasant sensation to a patient and minimizes friction between the head 114 and a patient's skin while dispensing the serum. Similarly, the configuration of the gearbox 134 will be apparent to one of ordinary skill in the art based on the function of the rotating wiper 138 described herein.

FIG. 2 also shows that the body 116 also houses a vacuum pipe 137 and a regulated feed pipe 140, which are connected to the vacuum connection 128 and the regulated supply connection 130, respectively. As shown, the regulated feed pipe 140 includes a feed control valve 142 operated by the feed control 120, enabling a user to control the flow of fluid or other material through the regulated feed pipe 140 as required in the operation of the automated multi-modality desincrustation and skin transdermal delivery treatment device 100. The feed control valve 142 shown is a one-way valve to prevent the backflow of material into the regulated feed pipe 140.

As further shown in FIG. 2, the body 116 houses a power switch 144 for turning on and off the motor 132. In the embodiment shown, the power switch 144 is adapted to be activated by the feed control 120, such that when the regulated feed pipe 140 is supplying material to the head 114, the motor 132 is activated to control the rotating wiper 138. However, it is understood that the power switch 144 and the feed control 120 may be independent or otherwise dependently configured. Accordingly, in some contemplated embodiments, the power switch 144 may be externally accessible.

The head contact switch 146 shown in FIG. 2 is a safety switch that prevents the gearbox 134 from engaging the motor 132 unless the head 114 is in contact with a treatment surface. For example, in the example shown, the rotating wiper 138 retreats into the head 114 approximately one millimeter when pressed against a surface to be treated. The movement of the rotating wiper 138 engages the head contact switch 146 to cause the gearbox 134 to engage the motor 132 to enable the motor 132 to drive the wiper drive 136 when the feed control 120 is activated. When the head 114 is removed from the treatment surface, the head contact switch 146 is deactivated and cuts the engagement between the motor 132 and the gearbox 134. As with the other elements described herein, the head contact switch 146 is a feature of the example shown in the illustrated figures, but the automated multi-modality desincrustation and skin transdermal delivery treatment device 100 may be designed to include fewer or additional features as will be apparent based on the description herein. For example, the head contact switch 146 may also prevent the operation of the feed pipe 140 or the stationary sprinkler base 152 when the rotating wiper 138 is not in contact with a treatment surface.

In the embodiment shown in FIG. 2, there is a vacuum chamber 148 within the head 114 for providing vacuum pressure surrounding the rotating wiper 138. The vacuum is provided in the vacuum chamber 148 via the vacuum pipe 137. As described further herein, the materials provided via the feed pipe 140 and the materials removed from the treated surfaces may be drawn into the vacuum chamber 148 and pulled through the hand tool 102 to the control base 104 to be collected in a waste receptacle (not shown) located, for example, in the supply drawer 112.

The extreme distal end of the hand tool 102 is defined by a treatment surface contacting tip 150. The tip 150 shown in FIG. 2 is a disposable and replaceable plastic semi-flexible that protrudes beyond the rotating wiper 138 such that the tip 150 is the only portion of the hand tool 102 that initially contacts the treatment surface. However, once the hand tool 102 is activated, the vacuum pressure pulls the treatment surface (i.e., a patient's skin) into contact with the rotating wiper 138 pushing the rotating wiper 138 into the head 114 activating the head contact switch 146 engaging the gearbox 134 causing the wiper drive 136 to drive the rotating wiper 138. It is understood that the tip 150 may be alternatively configured and adapted to work with various embodiments of the hand tool 102. The flexibility of the tip 150 helps improve the comfort level is using the device. When the tip 150 is able to provide an effective seal at the treatment site, the effects of the vacuum chamber 148 are improved. Replaceable tips 150 assist in providing longevity, comfort and cleanliness.

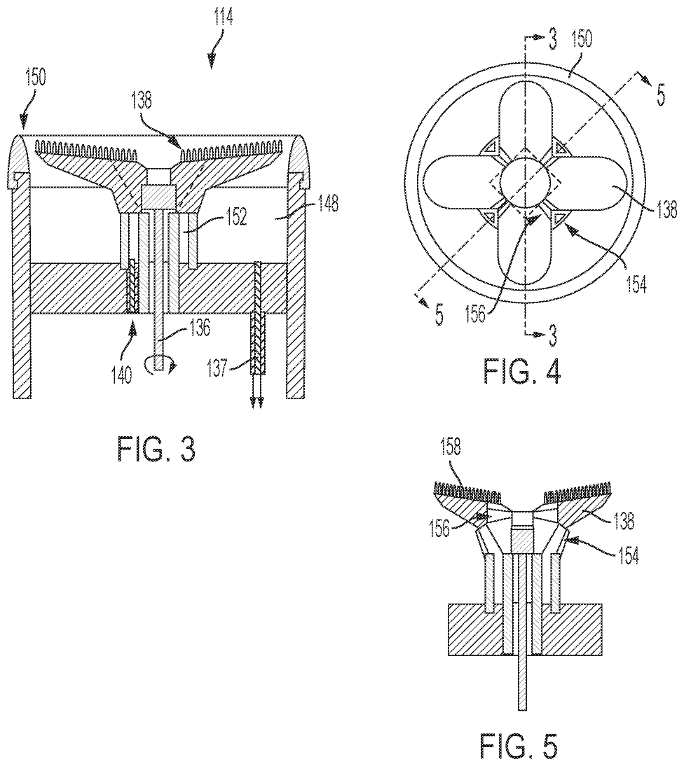

As further shown in FIG. 2, the feed pipe 140 supplies a stationary sprayer base 152 in the head 114 of the hand tool 102. The stationary sprayer base 152 interacts with the rotating wiper 138 as is described further with respect to FIG. 3.

Turning now to FIG. 3, the feed pipe 140 is shown to be in fluid communication with a stationary sprayer base 152. The stationary sprayer base 152 feed the material supply from the feed pipe 140 to the rotating wiper 138. As shown, the wiper drive 136 engages the rotating wiper 138 above the stationary sprayer base 152, enabling the rotating wiper 138 to rotate above the stationary sprayer base 152. The stationary sprayer base 152 feeds the material supply from the feed pipe 140 to a plurality of nozzles 154 in the rotating wiper 138, as shown in FIG. 4.

Turning now to FIG. 4, a top view of the rotating wiper 138 is shown illustrating the nozzles 154 as well as a plurality of drains 156. As shown in FIG. 4, the nozzles 154 are in fluid communication with the feed pipe 140 via the stationary sprayer base 152 enabling the supply material to transfer through the hand tool 102 to the treatment surface. In the example shown in FIG. 4, there are four nozzles 154, but it is understood that there may be any number of nozzles 154 provided in any configuration for accomplishing the goals of the subject matter disclosed herein. The drains 156 shown are in fluid communication with the vacuum chamber 148 via the rotating wiper 138. Accordingly, the materials provided through the nozzles 154 and the materials removed from the treatment surface may be collected through the drains 156 or around the exterior of the rotating wiper 138. Similar to the nozzles 154, the drains 156 may be provided in any number and configuration for accomplishing the goals of the subject matter disclosed herein

FIG. 5 shows a cross-sectional view of the rotating wiper 138 shown in FIG. 4. The cross-sectional view illustrates how the nozzles 154 communicate with the stationary sprayer base 152. As further shown in FIG. 5, the rotating wiper 138 may include an abrasive contact surface 158 for superficial abrasion and scrubbing of the treatment surface.

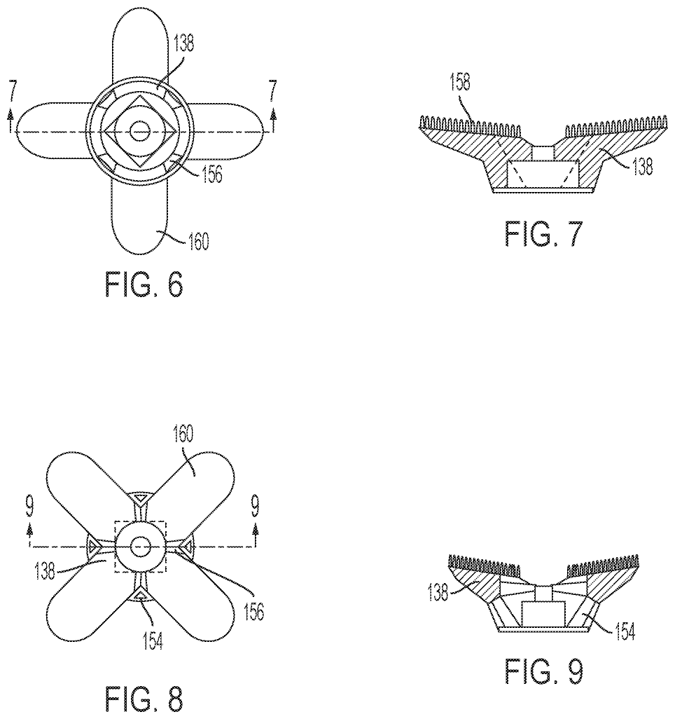

FIGS. 6-9 provide additional view of the rotating wiper 138 shown in FIG. 5. The rotating wiper 138 shown in FIGS. 6-9 is formed from silicone rubber and includes four wings 160. However, it is contemplated that the shape of the rotating wiper 138, the configuration of the contact surface 158 and the materials used may vary based on application. For example, it is contemplated that the contact surface 158 may be coated with and/or may comprise a more abrasive material. It is further contemplated that in some examples, the wings 160 may be replaced by a singular disc. Alternatively, there may be a greater or lesser number of wings 160 provided in varying sizes and shapes. As shown, the rotating wiper 138 is mounted onto the wiper drive 136 by a press fit, which secures the rotating wiper 138 to the wiper drive 136 by friction. The rotating wiper 138 may be easily replaced by pulling a used one off of the wiper drive 136 and pressing a new one in place.

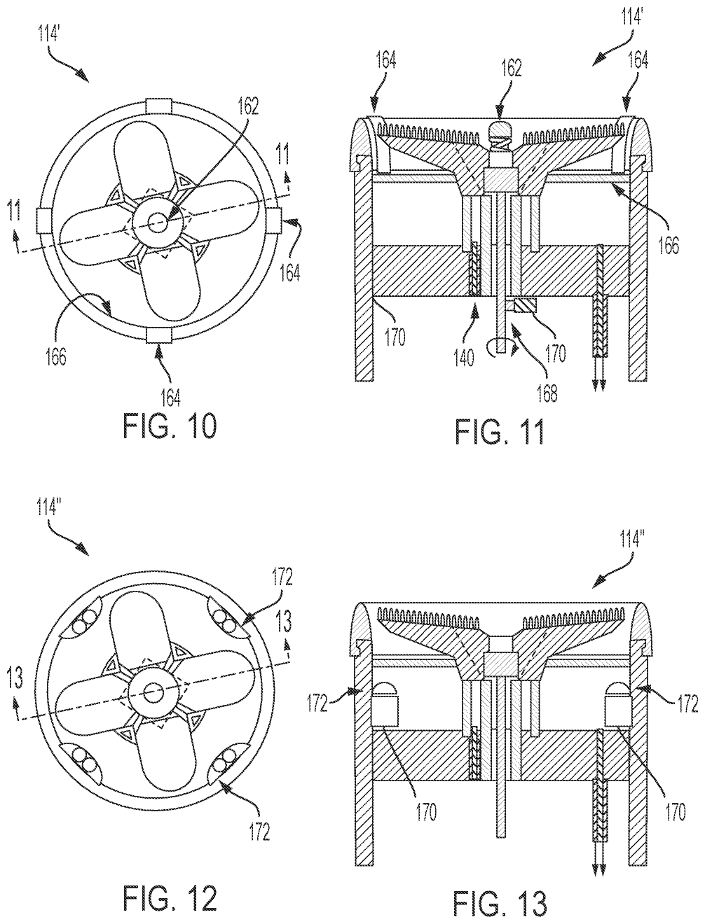

FIGS. 10-13 illustrate additional features and elements that may be incorporated into a head 114 of the automated multi-modality desincrustation and skin transdermal delivery treatment device 100. It is understood that the additional features and elements shown in these various embodiments are merely a subset of the various features and elements one may implement in an automated multi-modality desincrustation and skin transdermal delivery treatment device 100 to accomplish relevant treatments and procedures as will be understood by the disclosure provided herein.

In the example shown in FIGS. 10 and 11, a radio frequency (“RF”) wiper head 114′ is provided including a main electrode 162, four reference electrodes 164, a conductive band 166 in electrical contact with the reference electrodes 164, a brush contact 168 in electrical contact with the main electrode 162 and electrical leads 170 tying the conductive band 166 and the brush contact 168 into the electric and hydraulic lines 126 shown in FIG. 2. The brush contact 168 is used to provide an electrical contact through the rotating wiper drive 136. In other embodiments, other configurations for implementing the RF technology in the hand tool 102 as will be appreciated based on the disclosure herein.

The RF wiper head 114′ may be used to generate shallow to medium heat in the patient's skin along the treatment surface. For example, a vacuum may be applied to the treatment surface concurrently with energy generated by the RF module to warm the treated skin and assist in improving the efficacy of the treatments administered. Alternatively, the RF wiper head 114′ maybe used in an iontophoresis process, for example, serum droplets exiting the nozzles 154 may be positively charged by the main electrode 162 functioning as a cathode electrode with respect to the reference electrodes 164. Accordingly, the electric field will attract the droplets, causing them to accelerate and easing their penetration into the patient's skin. For example, it may be beneficial to provide an RF wiper head 114′ capable of generating approximately twenty watts at three MHz. In another example, the RF wiper head 114′ may be used for superficial skin tightening and lifting of the neck and face. In such case, the RF wiper head 114′ may be capable of generating approximately 50 watts at a frequency between, and including, 1 to 2.5 MHz.

In the example shown in FIGS. 12 and 13, an LED array head 114″ includes a light emitting diode (“LED”) array 172. Electricity is supplied to the LED array 172 via electrical leads 170 tying the LED array 172 into the electric and hydraulic lines 126 shown in FIG. 2. The LED array may include, for example, red and ultra-violet LED having independent electronic control, with each LED being optimized for appropriate treatment output levels. In one example, the output of the LED array may be used to encourage normal cell turnover and the production of collagen, such as, for example, one thousand mcd.

Although they are shown in separate independent examples, it is contemplated, for example, that the RF technology of the RF wiper head 114′ and the LED array 172 may be incorporated into a single head 114 and/or hand tool 102, providing a hand tool 102 with even greater functionality. In addition, the RF technology described herein and the LED array 172 may be incorporated into a hand tool 102 including any of the described heads 114.

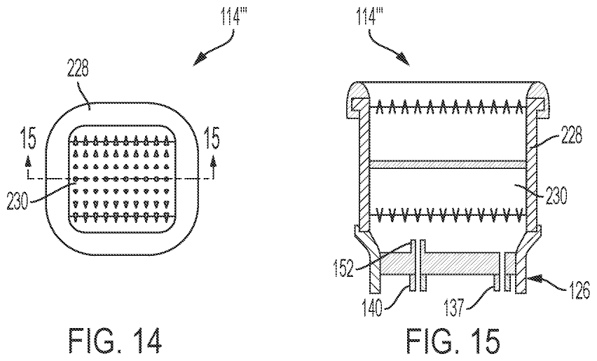

Referring to FIGS. 14 and 15, the skin transdermal delivery treatment device 100 may also accommodate a needle roller head 114′″. The needle roller head 114′″ includes an adaptor 226, a lid or casing 228, and a roller 230 for contacting the skin of the patient's face. In some embodiments, the casing 228 may be removeably attached to the adaptor 226 and the adaptor 226 may be removeably connected to the body 116. In other embodiments, the adaptor may be integrally formed with the casing 228. Alternatively, the adaptor 226 may not be a separate item but rather an extension of or part of the body 116. The adaptor 226 may be any suitable shape. The adapter 226 connects to the lid 228 through any suitable mechanism as is known in the art.

In one embodiment, the lid 228 may be detached from the adaptor 226 in order for a user to access the cavity formed from the adaptor 226 and the lid 228. For example, the lid 228 may be removed to provide a user with access to change the needle roller 230 to a different needle roller 230, for example, including a different needle density or needle thickness. In another example, the lid 228 may be removed to allow a user to exchange the roller 230 with the rotating wiper 138. The lid 228 may include an opening through which a plurality of needles 232 of the roller 230 may access a patient's skin. Typically, the lid 228 includes rounded edges and a smooth surface that may easily slide across a patient's skin.

The roller 230 is positioned inside a cavity that is formed from the adaptor 236 and lid 228 connection. For example, the roller 230 may be connected and stabilized to the adaptor 236 such that the roller 230 may spin without obstruction. Alternatively, the roller 230 may be stabilized and connected to the body 116. The roller 230 may include a plurality of needles 232 that protrude from an opening in the lid 228 when the skin transdermal delivery treatment device 220 is applied to a patient's skin. Alternatively, the skin may be pulled into the opening to access the plurality of needles 232. Movement of the roller 230 over a patient's skin causes the roller 230 to rotate, thereby bringing the needles 232 into contact with the patient's skin.

The needles 232 may be made of any suitable material, including titanium. The length of the needles 232 may be between, and including, 100 to 3000 micrometers, for example, 200 to 300 micrometers. In addition, the needles 232 may have a diameter between, and including, 0.10 to 0.20 millimeters, for example, 0.15 to 0.17 millimeters. The roller may be any suitable size, for example, the roller 230 may have a width between, and including, 15.0 and 25 millimeters, for example, 21.9 millimeters. The roller 230 may have a diameter between, and including, 15 and 30 millimeters, such as 20 millimeters. Typically, the roller contains between, and including, 125 to 500 needles, for example between, and including, 200 to 500 needles. Further, the needles 232 may be sterilized between use by gamma radiation, or other suitable means of sterilization.

As described above, the skin transdermal delivery treatment device 100 may further include at least one nozzle 154 for dispensing a liquid, such as a serum. The nozzle 154 may dispense the serum directly onto the roller 230. Alternatively, the nozzle 154 may be in fluid communication with an interior of the roller 230 such that the serum may be dispensed directly through the needles 232 or directly through openings in the surface of the roller 230. In other words, serum may be dispensed from the surface of the roller 230, through the needles 230, or through both.

The nozzle 154 may be in fluid communication with a serum source stored in a reservoir in the control unit 104. In one example, a serum source, such as a cartridge or container, may be housed in the body 116 and connected to the nozzle 154 for dispensing the serum. For example, once the serum source has been depleted, the serum source may be replaced with a new serum source for subsequent skin treatments. The serum source may be connected to a positive pressure source, such that the positive pressure pushes the serum out of the nozzle 154 and, in the example with hollow needles 232, out of the needles 232. In an example, the nozzle 154 may be designed such that the nozzle 154 only dispenses the serum when the roller 230 is rotating. As will be understood by those skilled in the art based on the description provided herein, the serum might be a liquid, gel, or other appropriate material.

In addition, the head 114′″ of the device 100 may be in fluid communication with a vacuum line 238 and/or a vacuum chamber, wherein the vacuum line 238 may be housed in the body 116. When the vacuum is on and the lid 228 is flesh against a patient's skin, a portion of the patient's skin may be pulled into the cavity housing the roller 230, wherein the patient's skin is penetrated by the needles 232 on the roller 230. The vacuum may also be used to remove excess serum from a patient's face after the roller 230 has passed over a certain area.

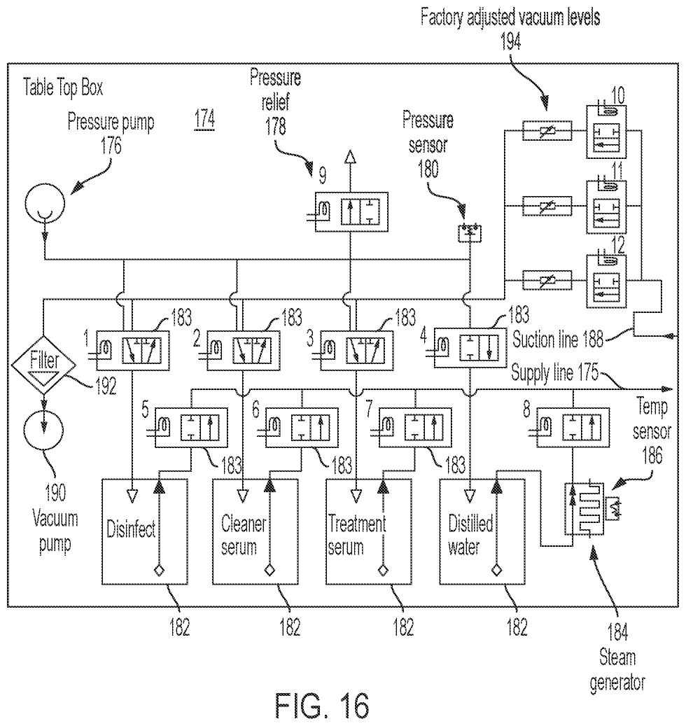

Turning now to FIG. 16, a schematic drawing of the hydraulic system 174 contained in the control unit 104 is shown. As shown in FIG. 16, the hydraulic system 174 includes a supply line 175 that ties into the regulated feed pipe 140 in the hand tool 102 for supplying various pressurized materials to the hand tool 102. In the example provided in FIG. 14, the supply line 175 is associated with a pressure pump 176, a pressure relief 178 and a pressure sensor 180 as will be understood by one of ordinary skill in the art based on the disclosure herein. In addition, the supply line 175 is fed by a plurality of reservoirs 182 containing various materials used in the operation of the automated multi-modality desincrustation and skin transdermal delivery treatment device 100. As shown, the associated valves 183 control the reservoirs 182. In an example, the supply line 175 is associated with two or more pressure pumps 176.

In one example, the reservoirs 182 may include a disinfectant reservoir 182, a cleaner serum reservoir 182, a treatment serum reservoir 182 and a distilled water reservoir 182. The distilled water reservoir 182 may be used, for example, to provide steam through the supply line 175 and, accordingly, may be associated with a steam generator 184 and a temperature sensor 186. It is contemplated that the number and type of reservoirs and associated components provided may vary depending on the materials and treatments provided by the automated multi-modality desincrustation, pollutant removal and skin transdermal delivery treatment device 100. The reservoirs 182 may be sealable and able to withstand the pressures applied in use of the automated multi-modality desincrustation and skin transdermal delivery treatment device 100. The reservoirs 182 may also include, for example, mechanical and/or electrical gauges for communicating the amount and the type of material held within.

As further shown in FIG. 16, the hydraulic system 174 includes a vacuum line 188 that ties into the vacuum pipe 137 in the hand tool 102 for supplying vacuum pressure to the head 114 of the hand tool 102. In the example provided in FIG. 16, the vacuum line 188 is associated with a vacuum pump 190, a filter 192 and various control valves 194 for providing different levels of vacuum pressure along the vacuum line 188.

The example shown in FIG. 16, is merely one example of the hydraulic system 174 contained within the control base 104, as will be understood by one of ordinary skill in the art based on the disclosure provided herein.

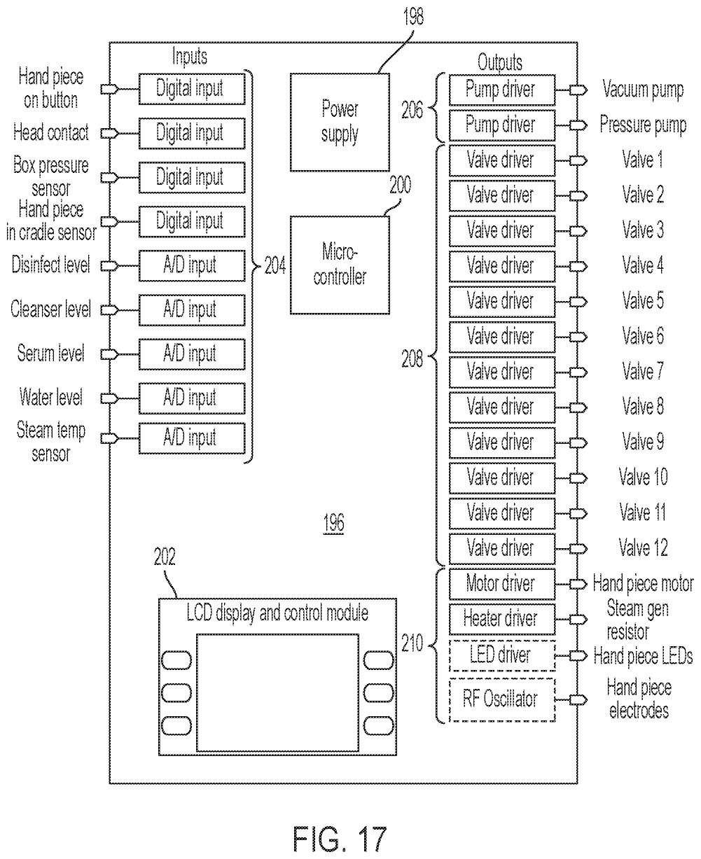

FIG. 17 is a schematic drawing of the electric system 196 contained in an example of the control base 104. As shown in FIG. 17, the electric system 196 includes a power supply 198, a controller 200, an LCD display and control module 202, a plurality of inputs 204, a plurality of pump drivers 206, a plurality of valve drivers 208 and additional drivers 210. As shown in the example shown in FIG. 17, the inputs 204 may be digital or analog to digital inputs depending on to which systems they are tied. Further, it is shown that there are a separate pump driver 206 for the pressure pump 176 and the vacuum pump 190, separate valve drivers for each valve, and separate additional drivers 210 for the motor 132, steam generator 184, LED array 172 and RF module.

The controller 200 shown in FIG. 17 runs application programs, accesses and stores data, etc. While further description of the controller 200 is provided below, it is understood that the controller 200 may be embodied in any one or more electronic systems arranged to control the electronic aspects of the automated multi-modality desincrustation and skin transdermal delivery treatment device 100 described herein.

Typically, the controller 200 is implemented by one or more programmable data processing devices. The hardware elements operating systems and programming languages of such devices are conventional in nature, and it is presumed that those skilled in the art are adequately familiar therewith.

For example, the controller 200 may be a central control processing system utilizing a central processing unit (CPU), memories and an interconnect bus. The CPU may contain a single microprocessor, or it may contain a plurality of microprocessors for configuring the CPU as a multi-processor system. The memories include a main memory, such as a dynamic random access memory (DRAM) and cache, as well as a read only memory, such as a PROM, an EPROM, a FLASH-EPROM, or the like. The system also includes mass storage devices when appropriate. In operation, the main memory stores at least portions of instructions for execution by the CPU and data for processing in accord with the executed instructions.

The controller 200 may also include one or more input/output interfaces for communications with one or more processing systems. Although not shown, one or more such interfaces may enable communications via a network, e.g., to enable sending and receiving instructions electronically. The physical communication links may be wired or wireless.

The controller 200 may further include appropriate input/output ports for interconnection with one or more output displays (e.g., monitors, printers, etc.) and one or more input mechanisms (e.g., keyboard, mouse, voice, touch, bioelectric devices, etc.) serving as one or more user interfaces for the controller 200. In the example shown, the controller 200 includes a graphics subsystem to drive the LCD display and control module 202. The links of the peripherals to the system may be wired connections or use wireless communications.

Those skilled in the art will recognize that the controller 200 also encompasses systems such as host computers, servers, workstations, network terminals, and the like. In fact, the use of the term controller 200 is intended to represent a broad category of components that are well known in the art. Accordingly, as one example, the control base 104 may be embodied, at least in part, in a personal computer.

Aspects of the automated multi-modality desincrustation and skin transdermal delivery treatment device 100 discussed herein encompass hardware and software for controlling the relevant functions. Software may take the form of code or executable instructions for causing a controller 200 or other programmable equipment to perform the relevant steps, where the code or instructions are carried by or otherwise embodied in a medium readable by the controller 200 or other machine. Instructions or code for implementing such operations may be in the form of computer instruction in any form (e.g., source code, object code, interpreted code, etc.) stored in or carried by any readable medium.

As used herein, terms such as computer or machine “readable medium” refer to any medium that participates in providing instructions to a processor for execution. Such a medium may take many forms, including but not limited to, tangible storage media, as well as carrier wave and tangible transmission media. Non-volatile storage media include, for example, optical or magnetic disks, such as any of the storage devices in any computer(s) shown in the drawings. Volatile storage media include dynamic memory, such as main memory of such a computer platform. Tangible transmission media include coaxial cables; copper wire and fiber optics, including the wires that comprise a bus within a computer system. Carrier-wave transmission media can take the form of electric or electromagnetic signals, or acoustic or light waves such as those generated during radio frequency (RF) and infrared (IR) data communications. Common forms of computer-readable media therefore include for example: a floppy disk, a flexible disk, hard disk, magnetic tape, any other magnetic medium, a CD-ROM, DVD, any other optical medium, punch cards paper tape, any other physical medium with patterns of holes, a RAM, a PROM and EPROM, a FLASH-EPROM, any other memory chip or cartridge, a carrier wave transporting data or instructions, cables or links transporting such a carrier wave, or any other medium from which a computer can read programming code and/or data. Many of these forms of computer readable media may be involved in carrying one or more sequences of one or more instructions to a processor for execution.

The automated multi-modality desincrustation and skin transdermal delivery treatment device 100 shown and described with respect to FIGS. 1-15 may be adapted to provide a number of treatments and applications. For example, the rotating wiper 138 may be used to remove pollutant from a treatment surface. A cleaning serum maybe applied to the treatment surface via the regulated feed pipe 140, the pollutant may be loosened from the skin by the cleaning serum and the rotating wiper 138 and the loosened materials may be pulled into the vacuum chamber 148. Further, steam may be applied to the treatment surface in addition to or in substitution for the cleaning serum. Additional treatments may be provided, for example, by providing one or more treatment serums through the regulated feed pipe 140, using the rotating wiper 138 to distribute the serum and removing the excess through the vacuum chamber 148.

The automated multi-modality desincrustation and skin transdermal delivery treatment device 100 may be used to perform a cleaning cycle. The cleaning cycle may be used, for example, between each change of materials to be passed through the regulated feed pipe 140. First, the cleaning cycle may begin by venting out pressure in the reservoir 182 from which material was last used. Then suction may be applied to the selected reservoir to cause the material to be pulled back into the reservoir. Finally, steam may be provided through the regulated feed pipe 140 to finish the cleaning cycle.

Similarly, a disinfecting cycle may be performed by injecting disinfectant material through the regulated supply line 140. The vacuum pipe 137 may be disinfected by pulling the disinfectant through the vacuum chamber 148 back through the hand tool 102.

The LCD display and control module 202 may be used as a user interface and control center. For example, menu options may be provided to a user through the LCD display and control module 202. Additional status information, treatments steps, instructions, error messages and other information may be provided to the user through the LCD display and control module 202. The LCD display and control module 202 is one example of a control screen 108 in a control base 104 as shown in FIG. 1. However, it is understood that various other displays may be used as the control screen 108.

Various treatments may be programmed into the automated multi-modality desincrustation and skin transdermal delivery treatment device 100. For example, the device 100 may provide a series of twenty to thirty minutes comprehensive, phased, aesthetic skin procedures utilizing the broad abilities of the automated multi-modality desincrustation and skin transdermal delivery treatment device 100. These procedures may be pre-programmed, including safety controls, to reach optimal results for the average skin type and conditions, for the operator to choose from a simple step-by-step graphic menu. Some of the applications may incorporate some or all of the following steps, in incremental or alternating phases: pollutant removal; cleaning and scrubbing of the skin; light abrasion of top epidermis layer; serum application of hydrating, rejuvenating, clearing cosmeceuticals; steam and vapor application; etc.

For example, one or more of the wiper head and the needle roller head includes one or more of one or more LEDs electrically coupled to a power source in the control unit through a power line in the hand tool, a radio-frequency generator electrically coupled to a power source in the control unit through a power line in the hand tool, and an electric field generator electrically coupled to a power source in the control unit through a power line in the hand tool, the electric field generator including a main electrode and one or more reference electrodes.

The control unit 104 includes a controller and memory, the memory storing program instructions that, when processed by the controller cause the controller to request a user selection of a treatment modality through the control screen, present instructions for use of the hand tool through the control screen, and automatically control one or more functions of the hand tool according to the selected treatment modality.

For example, the instructions may present a guide for a skin cleansing modality including instructing the attachment of the wiper head 114 to the body 102 and the control unit 104 adjusts a vacuum power applied through the wiper head 114 and provides a flow of cleansing material through the one or more nozzles 154 while rotating the wiper blades 138. The control unit 104 may subsequently provide water or steam through the one or more nozzles 154 while rotating the wiper blades 138. The control unit 104 may then subsequently provide a galvanic gel through the one or more nozzles 154 while rotating the wiper blades 138 and generating an electric field through the electric field generator.

Alternatively, the instructions may present a guide for a transdermal delivery treatment modality including instructing the attachment of a wiper head 114 including an abrasive contact surface 158 to the body 102 and the control unit 104 adjusts vacuum power and provides a flow of salicylic acid serum through the one or more nozzles 154 while rotating the wiper blades 138. The control unit 104 subsequently instructs the attachment of the needle roller head 104′″, adjusts vacuum power, and delivers a selected therapeutic serum through the one or more nozzles 154. The control unit 104 may then subsequently instruct the application of a bio cellulose mask, attachment of the LED array wiper head 114″, and activate the one or more LEDs 172.

Still further, the instructions may present a guide for a transdermal delivery treatment modality including instructing the attachment of a wiper head 114 including an abrasive contact surface 158 to the body 102 and the control unit 104 adjusts vacuum power and provides a flow of salicylic acid serum through the one or more nozzles 154 while rotating the wiper blades 138. The control unit 104 subsequently delivers a hydrating serum through the one or more nozzles 154 while rotating the wiper blades 138. The control unit 104 may then either subsequently instruct attachment of a further head 114′ or 114″ and activate the electric field generator or subsequently activate the radio-frequency generator.

The automated multi-modality desincrustation and skin transdermal delivery treatment device 100 described enables a user to direct the steam to the desired face and body areas, based on skin conditions. The device 100 provides a greater magnitude and intensity of skin infusion treatments, wherein the treatment device 100 enables expansion of clinical indications, particularly compared to fractional and non-fractional abrasive lasers, mesotherapy, and other invasive and non-invasive treatments. Particularly, when using a head 114 including a plurality of needles 232 that are capable of dispensing serums and gels, the device 100 may be used to replace the current mesotherapy techniques, and provide a device to treat cellulite reduction, stretch marks. In addition, the use of device 100 with a needle roller head 114 that may directly dispense serum into a patient's skin may also be used to administer growth hormones and hair restoration, e.g., androgenetic alopecia.

Using the heads 114, 114′, 114″, 114′″ described herein, the device 100 may be used to treat numerous skin conditions including, but not limited to, cellulite, stretch marks, scar reductions and preventions, and acne, among others. The device 100 may also be used to reduce age spots, blackheads, hyperpigmentation, discoloration due to sun damage, fine lines and wrinkles, crepey skin, surgical scars, enlarged pores, and acne scars. In addition, the device 100 may also be used to exfoliate skin and as part of anti-aging treatments.

It should be noted that various changes and modifications to the presently preferred embodiments described herein will be apparent to those skilled in the art. Such changes and modifications may be made without departing from the spirit and scope of the present invention and without diminishing its attendant advantages.

We claim:

1. A skin treatment device comprising:
a control unit including a control screen and a plurality of reservoirs; and
a hand tool connected to the control unit including:
a body with a base end and a head end, the body including:
a feed control valve controlling flow through a body feed line;
a body vacuum line; and
a motor;
the body further includes a control button configured to actuate the feed control valve;
a wiper head configured to selectively attach to the head end of the body, the wiper head including:
a wiper head casing including a fluid duct and a vacuum duct, wherein, when the wiper head is attached to the head end of the body, the liquid duct is in fluid communication with the body feed line and the vacuum duct is in fluid communication with the body vacuum line and vacuum duct;
a rotatable wiper structure including one or more wiper blades, wherein, when the wiper head is attached to the head end of the body, the rotatable wiper structure is operably driven by the motor; and
one or more nozzles in fluid communication with the plurality of reservoirs in the control unit through the body feed line and fluid duct; and
a needle roller head configured to selectively attach to the head end of the body, the needle roller head including:
a roller head casing including a fluid duct and a vacuum duct, wherein, when the wiper head is attached to the head end of the body, the liquid duct is in fluid communication with the body feed line and fluid duct and the vacuum duct is in fluid communication with the body vacuum line and vacuum duct;
a needle roller structure; and
one or more nozzles in fluid communication with the plurality of reservoirs in the control unit through the body feed line.

2. The skin treatment device of claim 1, wherein the wiper blades are silicone.

3. The skin treatment device of claim 1, wherein the wiper blades include an abrasive contact surface.

4. The skin treatment device of claim 1, wherein the wiper head includes one or more LEDs electrically coupled to a power source in the control unit through a power line in the hand tool.

5. The skin treatment device of claim 1, wherein the wiper head includes a radio-frequency generator electrically coupled to a power source in the control unit through a power line in the hand tool.

6. The skin treatment device of claim 1, wherein the wiper head includes an electric field generator electrically coupled to a power source in the control unit through a power line in the hand tool, the electric field generator including a main electrode and one or more reference electrodes.

7. The skin treatment device of claim 1, wherein the needle roller head includes one or more LEDs electrically coupled to a power source in the control unit through a power line in the hand tool.

8. The skin treatment device of claim 1, wherein the needle roller head includes a radio-frequency generator electrically coupled to a power source in the control unit through a power line in the hand tool.

9. The skin treatment device of claim 1, wherein the needle roller head includes an electric field generator electrically coupled to a power source in the control unit through a power line in the hand tool, the electric field generator including a main electrode and one or more reference electrodes.

10. The skin treatment device of claim 1, wherein one or more of the wiper head and the needle roller head includes one or more of:
one or more LEDs electrically coupled to a power source in the control unit through a power line in the hand tool;
a radio-frequency generator electrically coupled to a power source in the control unit through a power line in the hand tool; and
an electric field generator electrically coupled to a power source in the control unit through a power line in the hand tool, the electric field generator including a main electrode and one or more reference electrodes.

11. The skin treatment device of claim 10, wherein the control unit includes a controller and memory, the memory storing program instructions that, when processed by the controller causes the controller to:
request a user selection of a treatment modality through the control screen;
present instructions for use of the hand tool through the control screen; and
automatically control one or more functions of the hand tool according to the selected treatment modality.

12. The skin treatment device of claim 11, wherein the instructions present a guide for a skin cleansing modality including instructing the attachment of the wiper head to the body and the control unit adjusts a vacuum power applied through the wiper head and provides a flow of cleansing material through the one or more nozzles while rotating the wiper blades.

13. The skin treatment device of claim 12, wherein the control unit subsequently provides water or steam through the one or more nozzles while rotating the wiper blades.

14. The treatment device of claim 13, wherein the control unit subsequently provides a galvanic gel through the one or more nozzles while rotating the wiper blades and generating an electric field through the electric field generator.

15. The skin treatment device of claim 11, wherein the instructions present a guide for a transdermal delivery treatment modality including instructing the attachment of a wiper head including an abrasive contact surface to the body and the control unit adjusts vacuum power and provides a flow of salicylic acid serum through the one or more nozzles while rotating the wiper blades.

16. The skin treatment device of claim 15, wherein the control unit subsequently instructs the attachment of the needle roller head, adjusts vacuum power, and delivers a selected therapeutic serum through the one or more nozzles.

17. The skin treatment device of claim 16, wherein the control unit subsequently instructs the application of a bio cellulose mask and activates the one or more LEDs.

18. The skin treatment device of claim 15, wherein the control unit subsequently delivers a hydrating serum through the one or more nozzles while rotating the wiper blades.

19. The skin treatment device of claim 18, wherein the control unit subsequently activates the electric field generator.

20. The skin treatment device of claim 18, wherein the control unit subsequently activates the radio-frequency generator.
